(19) United States
(12) Patent Application Publication
(10) Pub. No.: US 2013/0149346 A1
(43) Pub. Date: Jun. 13, 2013

(54) DABIGATRAN ETEXILATE-CONTAINING PHARMACEUTICAL COMPOSITION

(71) Applicants: Dominique Meergans, Munich (DE); Jana Paetz, Bonn (DE)

(72) Inventors: Dominique Meergans, Munich (DE); Jana Paetz, Bonn (DE)

(73) Assignee: ratiopharm GmbH Graf-Arco-Strasse 3, Ulm (DE)

(21) Appl. No.: 13/583,273

(22) PCT Filed: Mar. 3, 2011
(86) PCT No.: PCT/EP11/53236

§ 371 Date: Oct. 15, 2012

(30) Foreign Application Priority Data
Mar. 8, 2010 (EP) 10155783.3

(51) Int. Cl. C07D 401/12

(52) U.S. Cl. CPC C07D 401/12
USPC 424/400; 546/273.4; 514/338

(57) ABSTRACT

The present invention relates to a pharmaceutical composition containing dabigatran etexilate or a pharmaceutically acceptable salt thereof as active ingredient.

The present invention relates to a pharmaceutical composition containing dabigatran etexilate or a pharmaceutically acceptable salt thereof as active ingredient.

Dabigatran etexilate (3-[(2-{[4-(hexyloxycarbonylamino-imino-methyl)-phenylamino]-methyl}-1-methyl-1H-benzimidazole-5-carbonyl)-pyridine-2-yl-amino]-propionic acid ethyl ester) has the following chemical formula:

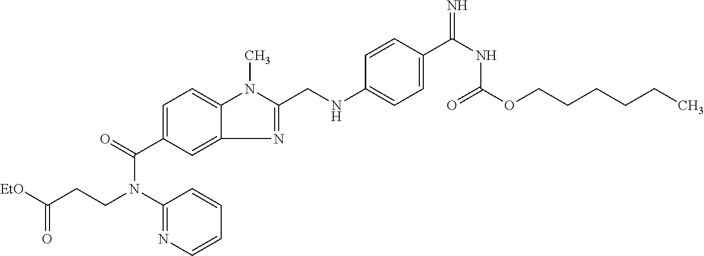 [SMILES: CCCCCCOC(=O)NC(=N)c1ccc(NCc2nc3cc(C(=O)N(CCC(=O)OCC)c4ccccn4)ccc3n2C)cc1]

This active ingredient is already known from WO 98/37075. The main indication field of said active ingredient is the postoperative prophylaxis of deep venous thromboses and the prophylaxis of strokes.

The solubility of the active ingredient in water is only 1.8 mg/ml. Moreover, the active ingredient has a strong pH-dependent solubility that is greatly increased in the acidic environment. This leads to the problem that conventional oral pharmaceutical compositions have large variations in the bioavailability since the solubility of the active ingredient depends on the pH value in the patient's stomach. This is particularly problematic with patients in whom the stomach pH value is changed by physiological variability, illness, or premedications (for example, PP inhibitors). There is therefore a need for oral pharmaceutical compositions of the active ingredient dabigatran etexilate that provide a release that is independent from the pH value of the stomach and thus, provide bioavailability of the active ingredient.

WO 03/074056 suggests a pharmaceutical composition for oral application that comprises in addition to the active ingredient one or more pharmaceutically acceptable organic acids having a water solubility of >1 g/250 ml at 20° C. However, the corresponding pharmaceutical compositions may cause incompatibilities in the patient, in particular if they already have a hyperacid stomach. Moreover, the addition of the organic acid restricts the possible amount of active ingredient in an appropriate tablet or capsule. This problem is further exacerbated by the fact that, as a rule, organic acids have only a low buffer capacity so that relatively large amounts of acid have to be added to cause a possible effect on the pH value of the ambience in dissolution of an appropriate tablet.

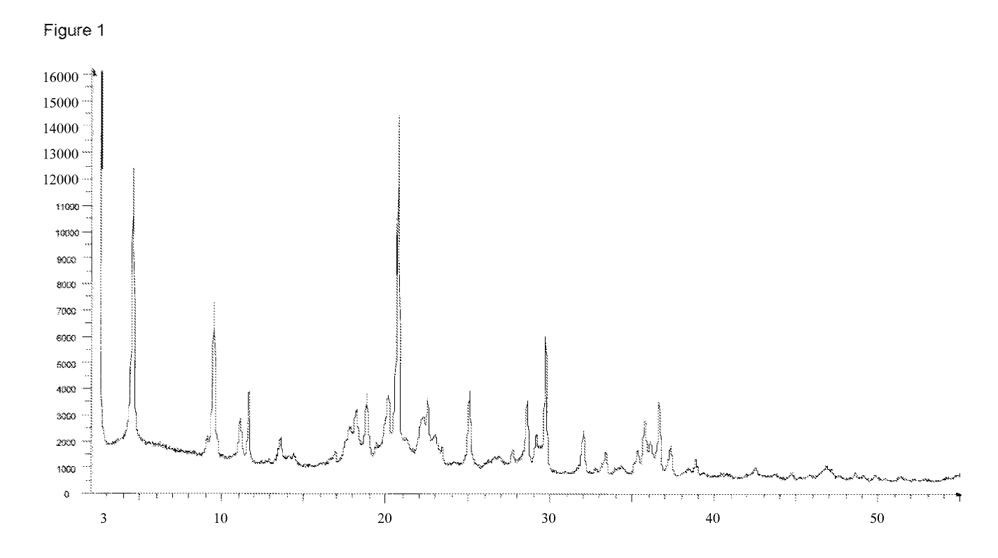

Moreover, WO 03/074056 describes pharmaceutical compositions that are prepared by spraying a dispersion of active ingredient and binder onto a core. According to the examples, here the ratio of active ingredient to binder is 5:1. The active ingredient is therefore used in large excess. Here, the active ingredient particles substantially remain and are still present in the crystalline form used. This is substantiated by the x-ray diffraction powder pattern of the correspondingly prepared market product Pradaxa® shown in FIG. 1. There can be seen the characteristic peaks of the crystalline dabigatran etexilate.

Other pharmaceutical formulations of the active ingredient dabigatran etexilate prepared by mixing or granulation of the components are known from WO 98/37075, WO 2006/114415, WO 2006/131491, and WO 2005/018615. In addition, WO 2005/028468 discloses various polymorphs of the crystalline dabigatran etexilate mesylate.

Finally, WO 2005/023249 discloses pharmaceutical compositions of the active ingredient dabigatran etexilate with a lipophilic, pharmaceutical acceptable, liquid, solid, or semi-solid carrier system. One method for the preparation of said pharmaceutical compositions is that a dispersion of active ingredient is prepared in the liquid (melted) carrier system under stirring. Under these conditions the active ingredient does not dissolve in the carrier system, but is present in the final composition as dispersion, i.e. as solid active ingredient particles embedded in the carrier system.

There still remains a need for pharmaceutical compositions providing the active ingredient dabigatran etexilate in a form having a pH value-independent release as far as possible, wherein the formulation does not contain an acid as pH value-regulating agent. Moreover, the formulation shall be easy to process and provide the active ingredient both in physically and chemically stable form.

It has now surprisingly been found that these and further problems can be solved in that the active ingredient dabigatran etexilate or a pharmaceutically acceptable salt thereof is provided in a non-crystalline form. Thus, the present invention relates to dabigatran etexilate or a pharmaceutically acceptable salt thereof in non-crystalline form.

A particularly suitable pharmaceutically acceptable salt of the dabigatran etexilate is the mesylate salt, i.e. the salt of the methanesulfonic acid.

Crystalline forms can be distinguished from non-crystalline forms of the active ingredient for example by DSC measurements or x-ray diffraction powder patterns. Crystalline active ingredient particles show characteristic peaks in the x-ray diffraction powder pattern (see, for example FIG. 1) that do not occur in the non-crystalline form of the active ingredient.

The present invention provides two different non-crystalline forms of dabigatran etexilate or a pharmaceutically acceptable salt thereof. In one embodiment it is a solid solution comprising a solid solvent and dabigatran etexilate or a pharmaceutically acceptable salt thereof dissolved therein. The second embodiment provides the active ingredient in an amorphous form, this form being provided as a composition with one or more hydrophilic polymers to be stable. The solid solution differs from the composition containing the amorphous form in that in the solid solution the active ingredient is present molecularly dispersed, whereas in the composition in addition to polymer particles there are present amorphous active ingredient particles. Thus, the solid solution represents a mixture on a molecular level, whereas the composition represents a mixture of macroscopic particles.

Accordingly, by a solid solution is herein understood a body that is solid at a temperature of 23° C. and a pressure of 101 kPa wherein the components are homogenously distributed. Accordingly, the active ingredient is molecularly dispersed in the solid solvent. Thus, the solid solution is substantially free of active ingredient particles wherein herein substantially free of active ingredient particles means that at least 80% by weight of the active ingredient are dissolved in the solvent, so is present molecularly dispersed, preferably at least 90% by weight, more preferred at least 95% by weight such as for example at least 99% by weight. Most preferred, the active ingredient is completely molecularly dispersed in the solvent. Any remaining active ingredient particles may be amorphous or crystalline. The molecular dispersion of the active ingredient and thus the presence of a solution can be characterized for example by electron micrographs, DSC measurements, or by a decrease of the peaks characteristic for the crystalline active ingredient particles in the x-ray diffraction powder pattern in dissolution of the crystalline active ingredient particles in the solvent.

Herein, by “solid solvent” are understood those solvents that are present as a solid at a temperature of 23° C. and a pressure of 101 kPa. Suitable solid solvents for the active ingredient dabigatran etexilate or a pharmaceutical acceptable salt thereof are for example hydrophilic polymers. These can be used alone or mixed with one or more polymers.

In general, the designation “hydrophilic polymer” comprises polymers with polar groups. Examples of polar groups are hydroxy, amino, carboxy, carbonyl, ethers, esters, and sulfonates. Hydroxy groups are particularly preferred.

Preferably, the hydrophilic polymer has a water solubility of >0.01 mg/ml at 23° C.

Depending on the subsequently described methods for the preparation of the solid solution also the melting point of the hydrophilic polymer may be relevant. In the preparation by melting the solid solvent the melting point of the hydrophilic polymer should be chosen such that it is below the melting point of the active ingredient to avoid an increased degradation of the active ingredient during processing. Preferably, the melting point of the hydrophilic polymer in this case should be <140° C., more preferred <120° C. For the preparation by spray drying the polymer can be selected regardless of its melting point.

Typically, the hydrophilic polymer has an average molecular weight in the range between 1000 and 250,000 g/mol, preferably 2000 and 100,000 g/mol, and particularly preferred between 4000 and 85,000 g/mol. Further, a 2% (w/w) solution of the hydrophilic polymer in pure water has preferably a viscosity between 0.2 and 18 mPas at 25° C. The viscosity is determined in accordance to the European Pharmacopoeia (Ph. Eur.), 6^{th} edition, section 2.2.10.

Further, the hydrophilic polymer has preferably a glass transition temperature (Tg) between 20° C. and 220° C., preferably 25° C. to 170° C. The glass transition temperature (Tg) is the temperature at which the hydrophilic polymer becomes brittle on cooling and soft on heating. That means that the hydrophilic polymer becomes soft above the glass transition temperature and can be plastically deformed without breaking. The glass transition temperature is determined by means of a Mettler-Toledo® DSC 1 using a heating rate of 10° C./min. and a cooling rate of 15° C./min.

Examples of suitable hydrophilic polymers are cellulose derivatives, in particular hydrophilic derivatives of the cellulose (e.g. hydroxypropylmethylcellulose (HPMC), carboxymethylcellulose, preferably as sodium or calcium salt, hydroxyethylcellulose (HEC), hydroxypropylcellulose (HPC)), starch, gum arabic, tragacanth gum, polyvinylpyrrolidone (PVP), preferably with a molecular weight of from 10,000 to 60,000 g/mol, copolymers of PVP, preferably co-polymers comprising vinylpyrrolidone and vinylacetate units (e.g. povidone, VA64, BASF), preferably with a molecular weight between 40,000 and 70,000 g/mol, poly(oxyethylene)alkyl ether, poly(oxyethylene) fatty acid esters (e.g. solutol), block copolymers of ethylene oxide and propylene oxide (poloxamer, pluronic), polymethacrylate derivatives, polyvinyl alcohols, polyvinyl alcohol derivatives, polyethylene glycols, and polyethylene glycol derivatives such as polyethylene glycol glycerides and fatty acid esters of polyethylene glycol as well as sucrose fatty acid esters.

Preferred hydrophilic polymers are polyethylene glycols, polyethylene glycol glycerides, block copolymers of ethylene oxide and propylene oxide, hydroxypropylmethylcellulose, and polyvinylpyrrolidone.

The weight ratio of active ingredient to solid solvent in the solid solution is not particularly restricted and can be freely chosen by the skilled person. However, it is important to choose at least as much solid solvent that the active ingredient is dissolved therein. The amount of the solid solvent needed depends i.a. on the manufacturing process used. So, for example by means of melt extrusion at relatively high temperatures and high pressure a larger amount of active ingredient can be dissolved in a given amount of solvent than in simply stirring the active ingredient into the melted solvent. However, if too much active ingredient is dissolved in the solvent there is the risk that the obtained solid solution is not sufficiently stable after cooling and that the active ingredient crystallizes out again. In general, the weight ratio of dabigatran etexilate or a pharmaceutically acceptable salt thereof to solid solvent in the solid solution should be ≦1:1, preferably ≦1:3 and preferably ≦1:5. For example, the weight ratio may be in the range of from 1:1 to 1:10, preferably in the range of from 1:1 to 1:6. However, also higher active ingredient loadings up to 10:1, preferably up to 5:1 are possible, in particular when preparing the solid solution as explained in more detail below by spray drying.

Moreover, the solid solution in addition to the solid solvent and the active ingredient can also contain further pharmaceutical acceptable excipients. Preferably, the solid solution additionally contains one or more crystallization inhibitors. These have beneficial effects on the long-term storage stability of the solid solution. Suitable crystallization inhibitors are inorganic or organic salts such as for example ammonium chloride. Also suitable as crystallization inhibitor is for example urea.

Other suitable excipients are for example the known antioxidants.

In one embodiment the solid solution according to the invention, in addition to the active ingredient and the solid solvent, does not contain any acidic components, in particular no organic and/or inorganic acids.

In particular, in the preparation of the solid solution according to the invention by melt extrusion that is subsequently described in more detail it may be of advantage if additionally higher-melting excipients having a surface >1.5 m²/g are added to the solid solvent. These counteract on a eutectic melting point of the final melts. Suitable for that are for example microcrystalline cellulose, highly-disperse silica, and calcium phosphate. Also combinations of lower and higher-melting polymers can be employed.

The solid solution according to the invention may be prepared by any known method for the preparation of appropriate solid solutions. For example, the active ingredient can be dissolved in a melt of the solid solvent and subsequently, the obtained solution can be cooled. Preferably, dissolution of the active ingredient in the melt of the solvent is carried out by melt extrusion. Then, if necessary, after cooling the obtained solid solutions can be further grinded, sieved, and either directly for example filled into capsules or first provided with additional excipients. Alternatively, the obtained melt granulate, either directly or together with further excipients, can be compressed to tablets.

It may be advantageous to prepare at first a pre-emulsion by using higher-melting polymers and to process it after lyophilization.

In an alternative embodiment, the solid solution according to the invention may be prepared by spray drying. For that, both the solid solvent and the active ingredient are dissolved in a further solvent, for example an ethanol/water mixture and subsequently spray dried in a known manner. Since both the solid solvent and the active ingredient at first are present dissolved the spray drying conditions may be chosen such that in the obtained granulate the active ingredient is present molecularly dispersed in the solid solvent so that the spray dried granulate represents the desired solid solution. One advantage of the spray drying method is that the polymer may be chosen regardless of its melting point.

The obtained spray dried granulate can in turn optionally after grinding and sieving either directly be filled into a capsule or provided with additional excipients. Alternatively, the spray dried granulate can be compressed to tablets either directly or together with further excipients.

The second embodiment of the present invention providing the dabigatran etexilate or a pharmaceutically acceptable salt thereof in a non-crystalline form is a composition comprising dabigatran etexilate or a pharmaceutically acceptable salt thereof in an amorphous form and one or more hydrophilic polymers. In contrast to the solid solution in addition to particles of the hydrophilic polymer in the composition amorphous active ingredient particles are present. The differences between the solid solution and the composition are seen for example in electron micrographs or can be determined by DSC measurements.

The composition according to the invention should be substantially free of crystalline active ingredient particles wherein by substantially free of crystalline active ingredient particles is meant that at least 80% by weight of the active ingredient are present in an amorphous form, preferably at least 90% by weight, more preferred at least 95% by weight such as for example at least 99% by weight. Most preferably, the active ingredient is completely amorphous.

In a further embodiment of the composition according to the invention the dabigatran etexilate or a pharmaceutically acceptable salt thereof is present in the composition in a particle size d(90) of less than 50 μm, preferably less than 30 μm, and most preferably less than 10 μm.

Suitable hydrophilic polymers for the composition according to the invention are those hydrophilic polymers described above in connection with the solid solution. Also the mentioned further excipients for the solid solution may be used for the composition according to the invention.

In general, the weight ratio of dabigatran etexilate or a pharmaceutically acceptable salt thereof to hydrophilic polymer in the composition according to the invention should be in the range of from 10:1 to 1:30, preferably in the range of from 10:1 to 1:10, and most preferably in the range of from 5:1 to 1:5.

It was surprisingly found that the composition according to the invention is obtainable by intensively grinding the active ingredient dabigatran etexilate or a pharmaceutically acceptable salt thereof with the hydrophilic polymer. Even if the active ingredient is employed in the crystalline form by grinding in the presence of the hydrophilic polymer an amorphization of the active ingredient particles takes place. Moreover, the hydrophilic polymer stabilizes the amorphous state of the active ingredient particles so that the composition obtained after grinding is both physically and chemically stable and can be processed to pharmaceutical compositions.

Thus, the present invention also relates to a method for the preparation of the composition described above which comprises grinding dabigatran etexilate or a pharmaceutically acceptable salt thereof in the presence of one or more hydrophilic polymers. Grinding can be carried out as dry or wet milling. Preferably, dry milling is carried out with cooling (e.g. with liquid nitrogen). Appropriate grinding processes and suitable mills are known to the skilled person.

For the stabilization of the micronized/amorphous phase of the active ingredient it may be of advantage if the composition in addition to active ingredient and hydrophilic polymer furthermore contains an emulsifying agent. Preferably, this is an emulsifying agent having a HLB value >12. Suitable emulsifying agents can be of natural or synthetic origin. For example, lecithin, sodium stearylsulfate, Tween 80, Mrij, and Brij are suitable.

The present invention also relates to pharmaceutical compositions comprising a solid solution or composition according to the invention as described above. For example, the pharmaceutical composition may be a tablet, capsule, sachet, powder, granulate, or pellet.

In addition to the present solid solvent or hydrophilic polymer the pharmaceutical composition can contain one or more further pharmaceutically acceptable excipients such as e.g. fillers, lubricants, flow control agents, release agents, and disintegrants. (“Lexikon der Hilfsstoffe für Pharmazie, Kosmetik and angrenzende Gebiete”, edited by H. P. Fiedler, 4^{th} edition and “Handbook of Pharmaceutical Excipients”, 3^{rd} edition, edited by Arthur H. Kibbe, American Pharmaceutical Association, Washington, USA, and Pharmaceutical Press, London).

Fillers: The pharmaceutical composition can contain one or more filler(s). In general, a filler is a substance that increases the bulk volume of the mixture and thus the size of the resulting dosage form. Preferred examples of fillers are lactose and calcium hydrogenphosphate. The filler may be present in an amount of 0 to 80% by weight, preferred between 10 and 60% by weight of the total weight of the composition.

Lubricants: The function of the lubricant is to ensure that the pelletizing and the ejection take place without much friction between the solids and the walls. Preferably, the lubricant is an alkaline-earth metal stearate or a fatty acid, such as stearic acid. Typically, the lubricant is present in an amount of 0 to 2% by weight, preferably between 0.5 and 1.5% by weight of the total weight of the pharmaceutical composition.

Disintegrants: Usually, by a disintegrant is meant a substance that is capable of breaking up the tablet into smaller pieces as soon as it is in contact with a liquid. Preferred disintegrants are croscarmellose sodium, sodium carboxymethyl starch, cross-linked polyvinylpyrrolidone (crospovidon) or sodium carboxymethyl glycolate (e.g. explotab) and sodium bicarbonate. Typically, the disintegrant is present in an amount of 0 to 20% by weight, preferably between 1 and 15% by weight of the total weight of the composition.

Flow control agents: As the flow control agent there can be used e.g. colloidal silica. Preferably the flow control agent is present in an amount of 0 to 8% by weight, more preferably in an amount between 0.1 and 3% by weight of the total weight of the composition.

Release agents: The release agent can be e.g. talcum and is present in an amount between 0 and 5% by weight, preferably in an amount between 0.5 and 3% by the weight of the composition.

The pharmaceutical compositions according to the invention have the advantage that the active ingredient is released from these in an essentially pH-independent manner. Without being bound by theory it is thought that this exceptional releasing behavior is ascribable to the combination of the non-crystalline form of the active ingredient with the presence of the solid solvent, in particular the hydrophilic polymer. It is thought that the active ingredient dissolves very quickly due to its non-crystalline form. Typically, here however the problem occurs that amorphous active ingredients often crystallize out again immediately after dissolution. Probably, this problem is solved by the presence of the hydrophilic polymer since the also dissolving hydrophilic polymer is possibly capable of stabilizing the oversaturated active ingredient solution in the direct environment of the pharmaceutical composition so that renewed crystallization is prevented.

The attached FIG. 1 shows the x-ray diffraction powder pattern of the market product Pradaxa®.

The present invention now is explained in more detail with respect to the following examples without interpreting them as being limiting.

In examples 1-6 the solid solution was prepared by melting the polymer, dissolving the active ingredient, adding the optionally present further excipients as well as subsequent cooling of the obtained melt.

In examples 7 and 8 the substances were dissolved in ethanol/water and subsequently spray dried (Büchi).

### EXAMPLE 1

| | |
|---|---|
| Dabigatran etexilate mesylate | 86.55 mg |
| PEG 6000 | 86.55 mg |

### EXAMPLE 2

| | | |
|---|---|---|
| Dabigatran etexilate mesylate | 86.55 | mg |
| Pluronic F127 | 432.75 | mg |

### EXAMPLE 3

| | | |
|---|---|---|
| Dabigatran etexilate mesylate | 86.55 | mg |
| Gelucire 50/13 | 86.55 | mg |
| Pluronic F127 | 12.5 | mg |

### EXAMPLE 4

| | | |
|---|---|---|
| Dabigatran etexilate mesylate | 86.55 | mg |
| Pluronic F68 | 25 | mg |

### EXAMPLE 5

| | | |
|---|---|---|
| Dabigatran etexilate mesylate | 86.55 | mg |
| Gelucire 55/13 | 432.75 | mg |
| Avicel 101 | 35.8 | mg |

### EXAMPLE 6

| | | |
|---|---|---|
| Dabigatran etexilate mesylate | 86.55 | mg |
| PEG 6000 | 86.55 | mg |
| Solutol HS | 3.0 | mg |

### EXAMPLE 7

| | | |
|---|---|---|
| Dabigatran etexilate mesylate | 86.55 | mg |
| HPMC (Pharmacoat 603) | 86.55 | mg |
| Solutol HS | 3.0 | mg |

### EXAMPLE 8

| | | |
|---|---|---|
| Dabigatran etexilate mesylate | 86.55 | mg |
| Povidon VA64 | 200.00 | mg |

### EXAMPLE 9

| | | |
|---|---|---|
| Dabigatran etexilate mesylate | 86.55 | mg |
| Pharmacoat 603 (HPMC) | 17.31 | mg |
| SDS | 4.32 | mg |

The active ingredient was grinded with HPMC and SDS on a Netzsch MicroCer at a number of revolutions of 3000/min. for 1½ h in water. The resulting suspension was either lyophilized, spray dried, or granulated onto a mixture of Avicel and HPMC.

### EXAMPLE 10

| | | |
|---|---|---|
| Dabigatran etexilate mesylate | 86.55 | mg |
| Sepitrab (Tween 80) | 437.75 | mg |

The mixture was grinded in a ball mill (Retsch) for 2 h.

### EXAMPLE 11

| | | |
|---|---|---|
| Dabigatran etexilate mesylate | 86.55 | mg |
| Lyocoat (pea starch) | 200.00 | mg |

The mixture was grinded in a ball mill (Retsch) for 2 h.

1. A non-crystalline form of dabigatran etexilate or a pharmaceutically acceptable salt thereof.

2. A solid solution comprising dabigatran etexilate or a pharmaceutically acceptable salt thereof dissolved in a solid solvent.

3. The solid solution according to claim 2, wherein the solid solvent comprises one or more hydrophilic polymers.

4. The solid solution according to claim 3, wherein one or more of the hydrophilic polymers has an average molecular weight ranging from 1,000 to 250,000 g/mol.

5. The solid solution according to claim 3, wherein one or more of the hydrophilic polymers has a glass transition temperature ranging from 20° C. to 220° C.

6. The solid solution according to claim 3, wherein the hydrophilic polymers are selected from the group consisting of cellulose derivatives, starch, gum arabic, tragacanth gum, polyvinylpyrrolidone, copolymers of the polyvinylpyrrolidone, poly(oxyethylene)alkyl ethers, poly(oxyethylene) fatty acid esters, block-copolymers of ethylene oxide and propylene oxide, poly(methacrylate) derivatives, polyvinyl alcohols, polyvinyl alcohol derivatives, polyethylene glycols, polyethylene glycol derivatives, and sucrose fatty acid esters.

7. The solid solution according to claim 6, wherein the hydrophilic polymers are selected from the group consisting of polyethylene glycols, polyethylene glycol glycerides, block-copolymers of ethylene oxide and propylene oxide, hydroxypropylmethylcellulose, and polyvinylpyrrolidone.

8. The solid solution according to claim 2, wherein the weight ratio of dabigatran etexilate or pharmaceutically acceptable salt to solid solvent is less than or equal to 1:1.

9. The solid solution according to claim 2, wherein the solution further comprises a crystallization inhibitor.

10. The solid solution according to claim 9, wherein the crystallization inhibitor is selected from the group consisting of ammonium chloride and urea.

11. A composition comprising an amorphous form of dabigatran etexilate or a pharmaceutically acceptable salt thereof in combination with one or more hydrophilic polymers.

12. The composition according to claim 11, wherein the dabigatran etexilate or pharmaceutically acceptable salt is present in the form of particles having a size d(90) of less than 50 μm.

13. The composition according to claim 11, wherein one or more of the hydrophilic polymers has an average molecular weight ranging from 1,000 to 250,000 g/mol.

14. The composition according to claim 11, wherein one or more of the hydrophilic polymers has a glass transition temperature ranging from 20° C. to 220° C.

15. The composition according to claim 11, wherein the hydrophilic polymers are selected from the group consisting of cellulose derivatives, starch, gum arabic, tragacanth gum, polyvinylpyrrolidone, copolymers of the polyvinylpyrrolidone, poly(oxyethylene)alkyl ethers, poly(oxyethylene) fatty acid esters, block-copolymers of ethylene oxide and propylene oxide, poly(methacrylate) derivatives, polyvinyl alcohols, polyvinyl alcohol derivatives, polyethylene glycols, polyethylene glycol derivatives, and sucrose fatty acid esters.

16. The composition according to claim 15, wherein the hydrophilic polymers are selected from the group consisting of hydroxypropylmethylcellulose, poly(oxyethylene) sorbitan mono-oleate, and starch.

17. The composition according to claim 11, wherein the weight ratio of dabigatran etexilate or pharmaceutically acceptable salt to hydrophilic polymer ranges from 10:1 to 1:30.

18. The composition according to claim 11, wherein the composition further comprises an emulsifying agent.

19. The composition according to claim 18, wherein the emulsifying agent is selected from the group consisting of lecithin, sodium stearyl sulfate, Tween 80, Mrij, and Brij.

20. A method for the preparation of a solid solution according to claim 2, said method comprising the step of dissolving dabigatran etexilate or a pharmaceutically acceptable salt thereof in a solid solvent.

21. The method according to claim 20, wherein the dissolution is carried out in a melt of the solvent.

22. The method according to claim 21, wherein the dissolution is carried out by means of melt extrusion.

23. The method according to claim 20, wherein the dissolution is carried out during spray drying of a solution of the solid solvent and dabigatran etexilate or a pharmaceutically acceptable salt thereof in a further solvent.

24. The method for the preparation of a composition according to claim 11, said method comprising the step of grinding dabigatran etexilate or a pharmaceutically acceptable salt thereof in the presence of one or more hydrophilic polymers.

25. A pharmaceutical composition comprising a solid solution according to claim 2 as active ingredient.

26. The pharmaceutical composition according to claim 25, wherein said composition is in the form of a tablet, capsule, sachet, powder, granulate, or pellet.

27. A pharmaceutical composition comprising a composition according to claim 11 as active ingredient.

28. The pharmaceutical composition according to claim 27, wherein said composition is in the form of a tablet, capsule, sachet, powder, granulate, or pellet.

29. The solid solution according to claim 2, wherein the weight ratio of dabigatran etexilate or pharmaceutically acceptable salt to solid solvent is less than or equal to 1:5.
